(19) United States
(12) Patent Application Publication
(10) Pub. No.: US 2024/0120037 A1
(43) Pub. Date: Apr. 11, 2024

(54) METHOD AND SYSTEM FOR HYBRID CLINICAL TRIAL DESIGN

(71) Applicant: Janssen Research & Development, LLC, Raritan, NJ (US)

(72) Inventor: Levon Demirdjian, San Diego, CA (US)

(73) Assignee: Janssen Research & Development, LLC., Raritan, NJ (US)

(21) Appl. No.: 18/376,817

(22) Filed: Oct. 4, 2023

Related U.S. Application Data
(60) Provisional application No. 63/414,898, filed on Oct. 10, 2022.

(51) Int. Cl. G16H 10/20; G16H 10/60; G16H 50/20

(52) U.S. Cl. CPC G16H 10/20; G16H 10/60; G16H 50/20

(57) ABSTRACT

Methods of designing a hybrid clinical trial including an external control arm (ECA) study located in at least one site, to support a randomized clinical trial (RCT) study for a treatment of a condition are disclosed. A Mahalanobis distance value is calculated based on a point comprising a set of values corresponding to a first plurality of feature variables corresponding to at least one ECA candidate; and a distribution of points comprising a set of values corresponding to the first plurality of feature variables of each of a plurality of RCT participants that have received the treatment. ECA candidates may be excluded as ECA participants if they are deemed outliers based on the Mahalanobis distance value. Recruitment is dynamically adjusted into at least one ECA participant site database by comparing sets of feature variables in at least one ECA participant site database to corresponding sets of feature variables in the RCT participant database.

### CROSS-REFERENCE TO RELATED APPLICATIONS

This application claims the benefit of U.S. Provisional Application No. 63/414,898 filed on Oct. 10, 2022. The contents of that application are incorporated by reference herein.

### FIELD OF THE INVENTION

This disclosure relates generally to designing and conducting human clinical trials involving real-world data.

### BACKGROUND

Controlled clinical trials have long been the gold standard for assessing the safety and efficacy of treatments. Now, medical researchers have access to hundreds of millions of real-world data (RWD) points about how people are using them, including electronic health records, health insurance claims and even mobile phone data. And, in some cases, these data points can be more useful to medical researchers than trial data. There are also situations in which reusing clinical trial data can reduce the need to conduct further trials. Researchers may also receive patient-level data, which is data about an individual person who is using a particular medication under investigation, for example. Researchers may also get access to aggregated data, which is a consolidation of data related to groups of people who use medications or treatments under investigation. Big data used in RWD may come from several sources, including insurance providers, as well as information that gets entered into electronic health records (EHR) when people visit healthcare providers, like physicians and nurse practitioners.

As patient privacy is of utmost importance, the data used in RWD studies may be key-coded so medical researchers cannot see anything that would allow someone to recognize the person, although the patient may be re-identified if needed. Sometimes ages or a zip code of persons may be visible, but the person's name, date of birth or Social Security numbers (and other patient-identifiable information) will not be visible.

Researchers may also use data for predictive modeling, which is trying to understand whether or which patients are responding the best or benefiting the most, and which groups might or might not be developing more side effects with the medicine. Real-world data can assist in better predicting outcomes for—groups of patients presenting with similar characteristics, thereby maximizing the benefits of a therapy and minimizing the risks. Predictive modeling using big data may go beyond just averages, to allow researchers to examine many more people with different backgrounds and baseline risk factors. This enables researchers to study the effectiveness and the safety of products among a diverse population.

Real-world data also allows researchers to take a closer look at populations that may not have been well represented in the original clinical trial and then study the impact of a particular treatment over a longer period of time. For example, pharmaceutical company Janssen developed a new model for translating RWD into real-world evidence (RWE) of health benefits by using de-identified patient information from four U.S. administrative claims databases as part of one of the largest and most comprehensive evaluations of the risk of hospitalization and other complications for certain type 2 diabetes treatments. See “Clinical Trials 2.0: 5 Ways Johnson & Johnson Is Helping Revamp—and Revolutionize—How They're Conducted”, by Hallie Levine, Sep. 2, 2019, published at https://www.jnj.com/innovation/5-ways-johnson-johnson-is-helping-revolutionize-clinical-trials.

Enacted in 2016, the 21st Century Cures Act expanded the use of real-world data (RWD) in the regulatory approval process. Life sciences companies are exploring how best to use RWD, including external control arm studies to support regulatory submissions. As mentioned above, the commonly accepted gold standard to evaluate efficacy of medical interventions is a randomized control trial or randomized clinical trial (RCT). In this method, researchers randomize study participants into two groups—one that receives intervention and one that does not. However, RCTs are time consuming, costly, and in some situations, not feasible. In an external control arm study, enrolled patients receiving the intervention are compared to patients outside of the study. The external control arm could be patients who received treatment earlier (historical) or a group treated at the same time but in a different setting (contemporaneous). Because some external control arm studies use RWD that is already collected, it can be an efficient way to evaluate the impact of an intervention.

In some situations, it can be relatively simple to create an external control arm. For example, if patients in the intervention arm have similar characteristics to those in historical RCT data, researchers can use the historical data to create an external control arm. Because these data must be tightly aligned, it may only be valid for small sub-populations.

Researchers could also use a microsimulation approach, in which models simulate disease progression, using RWD to tune parameters. These methods enable researchers to model long-term outcomes that might not be available in medical or claims data, but researchers must be rigorous in how they tune parameters and validate results.

When the external patient population differs from the trial participants, researchers need to use more advanced methods. For example, using RWD such as linked claims and electronic medical record data, researchers could identify a large cohort of patients diagnosed with a specific disease. These patients likely represent a broader range of disease severity and treatment patterns when compared to patients with the same diagnosis enrolled in a RCT. In these situations, researchers must use statistical matching and weighting methods to find a subset of patients that mirror the intervention group. The goal of these methods can help generate balanced pools of participants and estimate a variety of treatment effects. This type of study is likely the most common application of external control arm studies that would be utilized in regulatory submissions.

The next step for researchers is to evaluate study feasibility and methodology. During this phase, it is important to determine if the intervention arm and the external control arm of the study are appropriately aligned on patient selection criteria and baseline patient characteristics. This step requires evaluating potential data sources (e.g., insurance claims, medical records, linked claims-electronic medical records, patient registries) for use as the external control arm. Researchers should look for potential sources of bias between the different data sources. They should also select a data source for the external control arm that enables equal ascertainment of study outcomes in both arms of the study.

Researchers may rely on human clinical trials for information before products are approved, but rely on data after a product is approved to really understand its benefits in the “real world.” The data are complementary. However, RWD is generally available in the therapeutic area years after the new drug application (NDA) has been filed with the regulatory authorities such as the U.S. Food and Drug Administration.

The regulatory landscape for external control arm studies is new, complex and quickly evolving. Early conversations with regulatory authorities, coupled with a rigorous and well-designed external control arm strategy can help prepare life sciences organizations for this application of real-world data. Medical researchers may continue to engage with agencies and others to explore how hybrid clinical trial design can inform regulatory and clinical decisions. Thus, methods of designing hybrid clinical trials combining real-world data with clinical in an external control arm (ECA) study are needed.

### SUMMARY

This approach in clinical trial design and methodology could be used in any indication in support of the 21^{st} Century Cures Act and recent FDA guidance on RWD/RWE studies. The ECA developed in embodiments of the hybrid clinical trial design disclosed herein combines the use of both real-world data and clinical trial data in a hybrid approach, including both data captured in randomized clinical trials (RCT) and data from the real-world setting. Considering that clinical trials do not perfectly reflect practices and circumstances in the real-world setting and that data collected in the real-world setting may lack critical information needed for robust comparison of outcomes collected in the RCT, a hybrid approach is needed.

Moving the timeline to close the gap between agency approval and RWD availability would assist clinicians with providing effective and safe medical therapeutic pharmaceutical treatments. In one embodiment of the disclosed methods, the timeline for the ECA protocol may be moved into the Phase 3 of the FDA New Drug Application process, including the RCT collection of data. In addition to this, introducing a trifecta of outlier detection, dynamic adjustment of patient recruitment, and propensity score modeling (PSM) of the ECA (external control arm) to the randomized controlled trial (RCT) provides a novel approach to a hybrid clinical trial design.

In one embodiment of the disclosed methods, a safety comparison of orexin antagonist medication in the RCT to standard of care (SOC) in the ECA may be performed. However, there are inherent differences between the RCT and real-world cohorts (e.g., differences in healthcare seeking behaviors, baseline depression severity, etc.). To ensure that the comparison is valid, it is important to limit and control for the effects of measured confounders. A confounder, otherwise known as a confounding variable, confounding factor, or lurking variable, is a variable that influences both an independent variable and a dependent variable in an observational study and leads to a false correlation between the dependent and independent variables. Confounding is a problem common in observational studies.

Methods of designing a hybrid clinical trial including an external control arm (ECA) study located in at least one site, to support a randomized clinical trial (RCT) study for a treatment of a condition are disclosed. A Mahalanobis distance value is calculated based on a point comprising a set of values corresponding to a first plurality of feature variables corresponding to at least one ECA candidate; and a distribution of points comprising a set of values corresponding to the first plurality of feature variables of each of a plurality of RCT participants that have received the treatment. ECA candidates may be deemed outliers and excluded as ECA participants based on the Mahalanobis distance value. Recruitment is dynamically adjusted into at least one ECA participant site database by comparing sets of feature variables in at least one ECA participant site database to corresponding sets of feature variables in the RCT participant database.

### BRIEF DESCRIPTION OF THE DRAWINGS

FIG. 1 illustrates a computerized system in accordance with an embodiment of the present disclosure for developing a hybrid clinical trial design.

FIG. 2 illustrates one embodiment of a method of designing a hybrid clinical trial implemented using the computerized system of the embodiment of FIG. 1.

FIG. 3 illustrates further details of the outlier detection methods discussed in the embodiment of FIG. 2.

FIG. 4 illustrates an exemplary automated workflow for a hybrid clinical trial in accordance with an embodiment of the present disclosure.

FIG. 5 shows an example of a computer system, one or more of which may be used to implement one or more of the apparatuses, systems, and methods illustrated herein.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

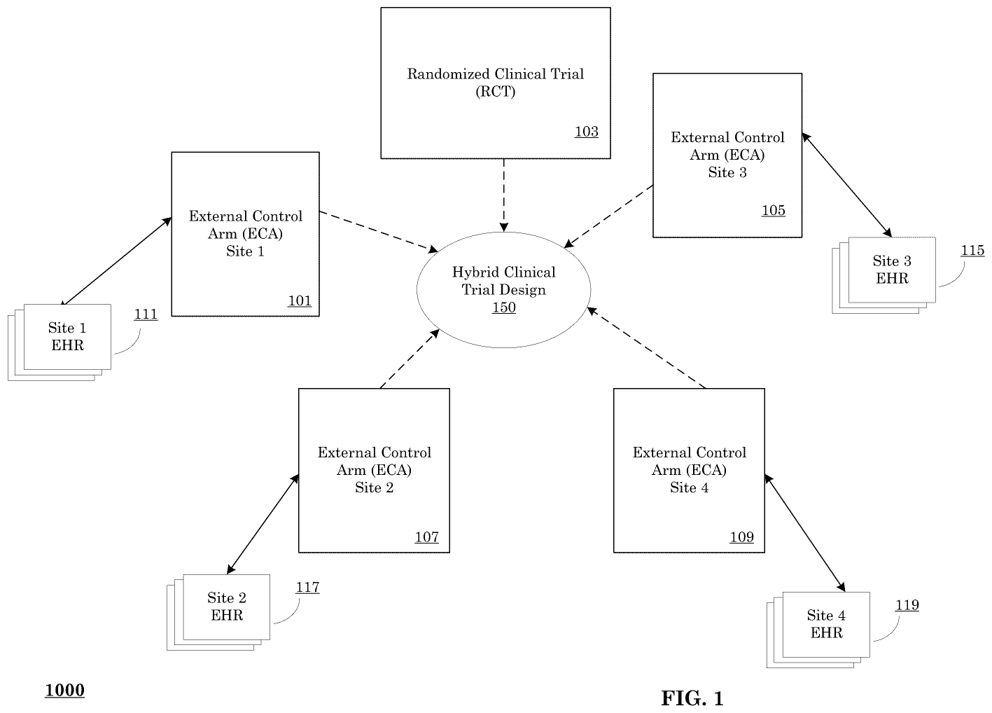

FIG. 1 illustrates a computerized system 1000 in accordance with an embodiment of the present disclosure for developing a hybrid clinical trial design. System 1000 is an example of one embodiment of the present invention.

System 1000 comprises a randomized clinical trial (RCT) 103, along with four external control arm (ECA) sites: ECA site 1 at 101, ECA site 2 at 107, ECA site 3 at 105, and ECA site 4 at 109. In one exemplary embodiment of the disclosed systems and methods, there are 4 ECA study sites as shown in FIG. 1. Other inventive embodiments of the systems and methods disclosed herein may include less than 4 ECA sites, such as one, two or three ECA study sites, or more than 4 ECA study sites, such as five, six, or up to ten or more ECA study sites.

ECA sites 1, 2, 3 and 4 are each operably connected to a site-specific electronic health records (EHR) database which are used as a source of information for candidates for participants in the ECA study at each site: Site 1 EHR 111 for ECA Site 1 101, Site 2 EHR 117 for ECA Site 2 107, Site 3 EHR 115 for ECA Site 3 105, and Site 4 EHR 119 for ECA Site 4 109. The 4 ECA sites located at 101, 105, 107 and 109, coupled with the RCT 103, all contribute data to the Hybrid Clinical Trial Design 150 as shown in FIG. 1 herein.

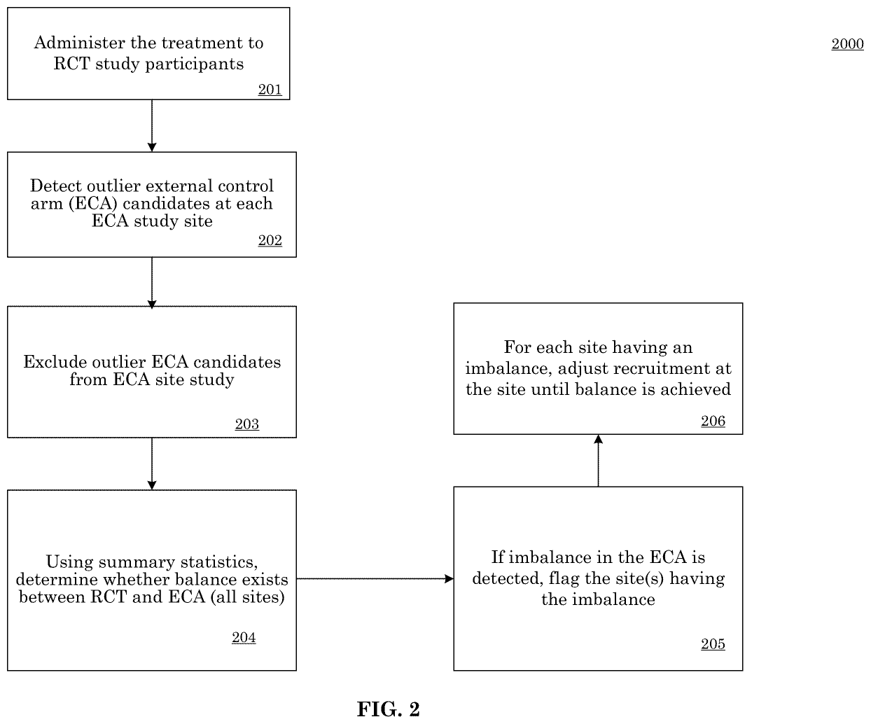

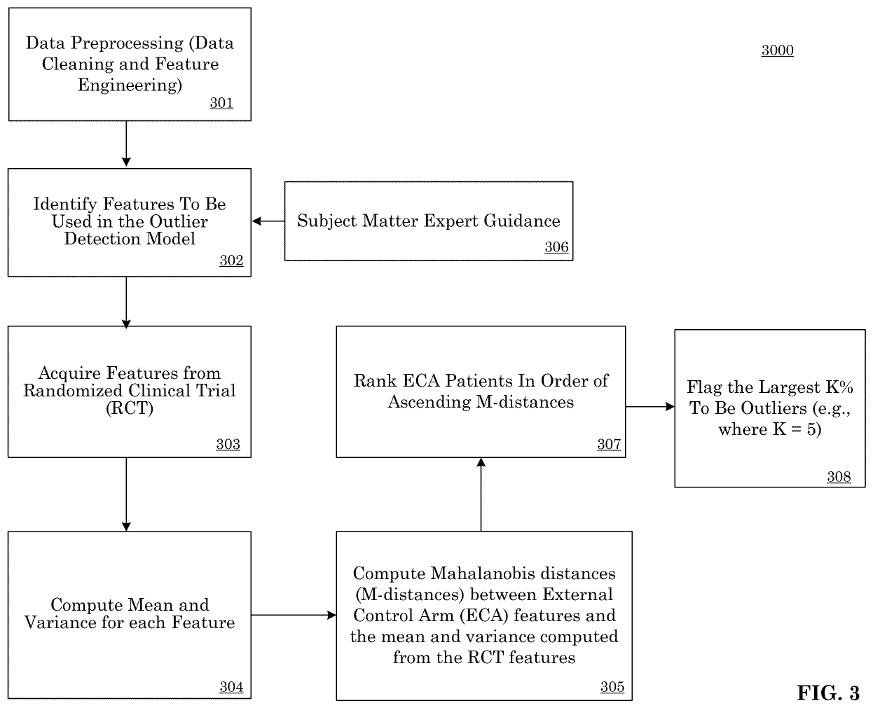

FIG. 2 illustrates one embodiment of a method 2000 of designing a hybrid clinical trial implemented using the computerized system of the embodiment of FIG. 1. Step 201 administers the treatment (e.g., RCT study drug or intervention) to RCT study participants. In one embodiment of the disclosed methods and systems, the RCT study participants are individual persons. Step 202 detects outlier external control arm (ECA) candidates at each ECA study site. Further description of the methodologies and computerized systems used to perform this detection step 202 is provided in FIG. 3.

At step 203, the outlier ECA candidates identified in step 202 are excluded from enrolling into the ECA study at each study site. At step 204, summary statistics are used to determine whether balance exists between one or more feature variables of each RCT participant and the collective ECA study sites, wherein the ECA study results are calculated using summarized data pooled across all of the ECA study sites. At step 205, if an imbalance is detected between the RCT participant statistics and the ECA statistics calculated using the collective data pooled across all of the ECA study sites, the individual ECA study site or sites having the imbalance is flagged.

At step 206, for each ECA site where an imbalance is noted, recruitment is adjusted until overall balance of the statistics for the evaluated features of the RCT study participants with statistics for the evaluated features of all ECA study participants across all ECA sites is achieved.

FIG. 3 illustrates further details of the outlier detection methods discussed in the embodiment of FIG. 2. In step 301, data preprocessing, including data cleaning and feature engineering, on the EHR data, or any patient-provided data from each of the ECA sites are performed. Feature engineering as discussed with respect to embodiments of the disclosed invention is the process of selecting, manipulating, and transforming raw data into features that can be used in subsequent analysis. Features are characteristics of patients that are measured at baseline before they receive study treatment. More generally, for outlier detection, it may be possible to remove the requirement that features need to be measured at baseline, as this may not typically be done, or may not be feasible. However, it is important to account for differences in the two populations that are not due to study treatment.

In step 302, one or more features used in the outlier detection model are identified. Features that are commonly extractable for electronic health records (EHR) data, such as age, gender, race, ethnicity, etc., may be used, including any other available EHR data that may be considered clinically important for the investigation. In some embodiments of the disclosed invention, six or more different features may be investigated. In other embodiments of the disclosed invention, up to 16 or 17 different measurements may be investigated for both the ECA and RCT enrolled patients, although more or fewer measurements or features may of course be investigated as determined by appropriate subject matter experts and/or the study investigators. In addition, for embodiments of the disclosed invention, the data is agnostic to the EHR system of the site and is not EHR-specific, as long as the data may be translated to a common format such as an Excel spreadsheet or other data storage and organization format known in the art. Such a format may also be applied to other features that may not necessarily be available in EHR data, but may also be considered valuable for study depending on the type of study or health intervention under investigation. For example, for a pharmaceutical intervention to treat depression, features such as smoking status, number of lifetime depression episodes, baseline depression severity, mental health or psychiatric health history, or history or current status of substance abuse may be considered relevant for analysis. This type of data may include non-EHR data such as may be available in a clinical database or a patient-reported outcomes (PRO) database. Subject matter guidance (e.g., from study investigators, researchers, or physicians) may be provided to select the appropriate features at step 303, that are identified as typical or important by clinical experts, in order to ensure that compatible patients are enrolled for both the RCT and ECA for the three phases of the study.

At step 304, a mean and variance calculation is performed for the data of each feature under investigation of the RCT study participants. Next, at step 305, a Mahalanobis distance (M-distance) is calculated between the corresponding feature data of each patient in the external control arm (ECA) study and the mean and variance computed for the corresponding feature from the RCT study participants. As is known to those skilled in the art, Mahalanobis distance is the distance between two points in multivariate space. In a regular Euclidean space, variables (e.g., x, y, z) are represented by axes drawn at right angles to each other, and the distance between two points may be measured with a ruler. For uncorrelated variables, the Euclidean distance equals the M-distance. However, if two or more variables are correlated, the axes are no longer at right angles, and the measurement becomes impossible with a ruler. In addition, if there are three or more variables, the points cannot be plotted in regular 3D space at all. M-distances solve this problem, as it measures distances between points, even correlated points for multiple variables (here, features of the RCT participants). The Mahalanobis distance measures distance relative to the centroid—a base or central point which can be thought of as an overall mean for multivariate data. The centroid is a point in multivariate space where all means from all variables intersect. The larger the M-distance, the further away from the centroid the data point is. A common use for the M-distance is to find multivariate outliers, which indicates unusual combinations of two or more variables. See Stephanie Glen. “Mahalanobis Distance: Simple Definition, Examples” from StatisticsHowTo.com: Elementary Statistics for the rest of us!; https://www.statisticshowto.com/mahalanobis-M-distances are included with many well-known and commonly used statistics packages, such as IBM's Statistical Product and Service Solution (SPSS), Minitab, Stata, and SAS/STAT. A definition of M-distance is available at Varmuza, K. & Filzmoser, P., Introduction to Multivariate Statistical Analysis in Chemometrics. CRC Press, 2016, p. 46.

At step 307, the ECA study patients may be ranked in the order of ascending M-distances. At step 308, the largest K % of the M-distances may be considered as outliers. In one embodiment of the disclosed invention, K=5, although in other embodiments other values of K may be used such as K=10. In some embodiments of the invention disclosed herein, the value of K selected may also determine the selection of sample size of an RCT study. This selection of sample size depends on the specific use case and threshold at which the study team are classifying outliers. For example, if the study team wishes to consider the top 10% of values to be outliers, then at least 10 samples are needed. If the study team wishes to consider the top 5% of values to be outliers, then at least 20 samples are needed. In one embodiment of the disclosed invention, at least 20 patients are required, although many more patients could certainly be examined (and may also provide for a more statistically robust investigative analysis).

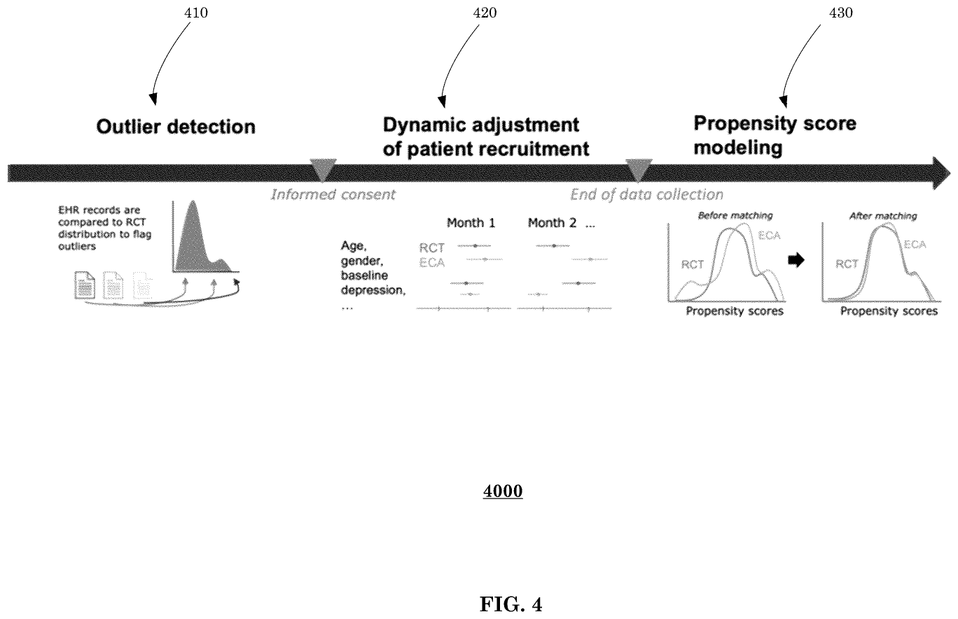

FIG. 4 illustrates an exemplary automated workflow 4000 for a hybrid clinical trial in accordance with an embodiment of the present disclosure. Workflow phase 410 sets forth an outlier detection phase, where EHR records are compared to the RCT sample distribution to flag outliers as described above in detail with respect to FIG. 3, in order to determine whether a particular individual person should or should not be recruited into the ECA portion of the hybrid trial.

Workflow phase 420 set forth a phase of dynamic adjustment of patient recruitment, where recruitment is adjusted into the ECA portion of the study. In some embodiments of the disclosed invention, dynamic recruitment adjustment may be performed at regular time intervals, such as monthly. More frequent (e.g., weekly or bi-weekly) or less frequent (e.g., bi-monthly or quarterly) dynamic recruitment adjustments may be performed and contemplated within the scope of the disclosed invention, based on the needs of the particular study under investigation.

Imbalances may be identified between the RCT and ECA portions of the study under investigation as follows. Imbalances in specific features (e.g., age) are identified using a standard metric called the absolute standardized mean difference (aSMD). In one embodiment of the disclosed invention, a feature that has an aSMD>0.10 between the RCT (in the case of the safety comparison of an orexin antagonist medication to the standard of care, the orexin antagonist medication would be the study intervention in the RCT) and the pooled ECA (across all ECA sites in the study investigation) will be considered to be imbalanced. In other embodiments of the invention, aSMD values may have a higher (such as 0.25) or lower threshold to be considered as an imbalance. In some embodiments of the disclosed invention, the RCT feature is compared to the pooled ECA sites since an imbalance between the RCT and one site may be offset by a balance or imbalance at another site. The interest is in joint balance, i.e., balance between the RCT and all of the ECA sites pooled together. The ECA data (patient level data) is used to detect an imbalance against the RCT. If there is an imbalance against the RCT, then recruitment into the ECA is adjusted accordingly. Recruitment is adjusted only when there is imbalance across all sites pooled (so there can be imbalance at one site, but as long as the other sites “compensate” then that is acceptable).

Measurement of the aSMD as determined in some embodiments of the present invention may be performed in accordance with methods known by those investigators skilled in the art, including for example methodologies described in Stuart, E. A. et at, “Prognostic score-based balance measures can be a useful diagnostic for propensity score methods in comparative effectiveness research”, Journal of Clinical Epidemiology 66 (2013) S84-S90. (doi: doi.org/10.1016/j.jclinepi.2013.01.013)

Dynamic adjustment of recruitment into the ECA sites to accomplish the interim balancing across the pooled ECA sites is performed in some embodiments of the present invention by having a principal investigator or other study responsible researcher or physician contacting the ECA sites where imbalances are flagged (e.g., by examining metrics such as the M-distance or aSMD values for specific sites or individual patients or participants at the specific ECA site), and working with a principal investigator at that site to see how recruitment may be adjusted in order to achieve balance. For example, if the ECA site is recruiting younger patients than what is seen in the RCT participant population, the ECA site may be advised to start recruiting older patients into the ECA at that site.

Workflow phase 430 as shown in FIG. 4 is propensity score modeling. In one embodiment of the disclosed invention, propensity score modeling takes place at the end of the data collection phase of the ECA and RCT. In other embodiments of the disclosed invention, the propensity score modeling will take place after enrollment of participants into the ECA and RCT studies has been completed and can occur concurrently with at least a portion of the data collection phase. In addition, in some embodiments of the disclosed invention, patients that were enrolled into the ECA, but subsequently dropped out or were lost to follow-up may still be included in the propensity score modeling phase. Propensity scores may be estimated using any of a variety of methods known to data analysts skilled in the art, such as logistic regression. Other methods may include machine learning-based models, probit models, generalized boosted models, discriminant analysis, neural networks, or classification trees.). See, e.g., Westreich, Daniel et al. “Propensity score estimation: neural networks, support vector machines, decision trees (CART), and meta-classifiers as alternatives to logistic regression.” Journal of Clinical Epidemiology vol. 63, 8 (2010): 826-33. (doi: doi.org/10.1016/j.jclinepi.2009.11.020).

Propensity scores are bounded between 0 & 1. A propensity score value close to 1 corresponds to a propensity score model being confident that the person is enrolled in the RCT (even if they are actually enrolled in the ECA); conversely, a value close to 0 corresponds to the model being confident that the person is enrolled in the ECA (even if they are actually enrolled in the RCT). Individuals with propensity score values close to 0.5 are individuals that the propensity score model has difficulty distinguishing as being enrolled in the ECA or RCT (i.e., from the models point of view, they are seen as close to equally likely to having been enrolled in either the RCT or RCT).

Propensity score models operate under the assumption that given an individual exposed to the treatment under investigation and an individual unexposed to the treatment under investigation, where both have the same (or nearly the same) propensity score, treatment assignment for these two individuals is independent of all confounding factors. Under this assumption of no unobserved or unmeasured confounding, matching exposed and unexposed individuals in a cohort will allow the data analyst to obtain an unbiased estimate of the average causal effect of the treatment on the outcome. See, e.g., Westreich, et al. referenced above. In addition to matching propensity scores, weighting using propensity scores may also be used to determine that any effects of confounding differences between the ECA and RCT study cohorts are limited and controlled for. One weighting scheme used in some embodiments of the disclosed invention proceeds as follows: for each individual, compute their estimated propensity score (PS), and use the following weights: Weight=1/PS for RCT participants, and Weight=1/(1−PS) for ECA participants, in a weighted regression of outcomes. This is referred to as inverse-probability of treatment weighing (IPTW). Other weighting schemes that are contemplated as within the scope of the invention disclosed herein include: stabilized IPTW (sIPTW): P(RCT)*(1/PS) for RCT, (1−P(RCT))/(1−PS) for ECA, where P(RCT) is the probability of being in the randomized clinical trial (versus the probability of being in the external control arm); and overlap weights: (1−PS) for RCT, PS for ECA. Overlap weighting is further described for some embodiments of the invention in the following reference which is incorporated herein in its entirety: Thomas L E, Li F, Pencina M J. Overlap Weighting: A Propensity Score Method That Mimics Attributes of a Randomized Clinical Trial. JAMA. 2020; 323(23):2417-2418. (doi:doi.org/10.1001/jama.2020.7819).

The patient's data is being weighted by the propensity score (for example, the patient data is being weighted proportional to 1/PS if IPTW is used). These weights are automatically determined once it is determined which weighting scheme to use.

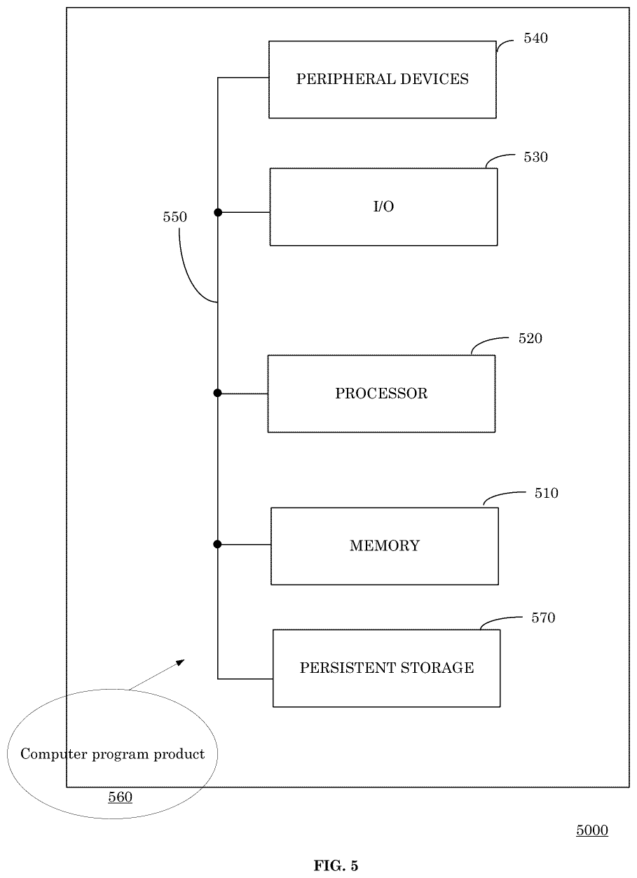

FIG. 5 shows an example of a computer system 5000, one or more of which may be used to implement one or more of the apparatuses, systems, and methods illustrated herein, such as system 1000 and methods 2000, 3000 and 4000. Computer system 5000 executes instruction code contained in a computer program product 560. Computer program product 560 comprises executable code in an electronically readable medium that may instruct one or more computers such as computer system 5000 to perform processing that accomplishes the exemplary method steps performed by the embodiments referenced herein.

The electronically readable medium may be any non-transitory medium that stores information electronically and may be accessed locally or remotely, for example via a network connection. In alternative embodiments, the medium may be transitory. The medium may include a plurality of geographically dispersed media each configured to store various parts of the executable code at different locations and/or at different times. The executable instruction code in an electronically readable medium directs the illustrated computer system 5000 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 560 may reside in one or more storage persistent media (not separately shown) communicatively coupled to system 5000 for loading and storage in persistent storage device 570 and/or memory 510 for execution by processor 520. Computer system 500 also includes I/O subsystem 530 and peripheral devices 540. I/O subsystem 530, peripheral devices 540, processor 520, memory 510, and persistent storage device 570 are coupled via bus 550. Like persistent storage device 570 and any other persistent storage that might contain computer program product 560, memory 510 is a non-transitory media (even if implemented as a typical volatile computer memory device). Moreover, those skilled in the art will appreciate that in addition to storing computer program product 560 for carrying out processing described herein, memory 510 and/or persistent storage device 570 may be configured to store the various data elements referenced and illustrated herein.

Those skilled in the art will appreciate computer system 5000 illustrates just one example of a system in which a computer program product in accordance with an embodiment of the present invention may be implemented. To cite but one example of an alternative embodiment, execution of instructions contained in a computer program product in accordance with an embodiment of the present invention may be distributed over multiple computers, such as, for example, over the computers of a distributed computing network.

Instructions for implementing a machine learning-based propensity score model, a logistic regression model, a probit model, and/or an artificial neural network implementing any of the above in accordance with disclosed embodiments may reside in computer program product 560. When processor 520 is executing the instructions of computer program product 560, the instructions, or a portion thereof, are typically loaded into working memory 510 from which the instructions are readily accessed by processor 520.

In one embodiment, processor 520 in fact comprises multiple processors which may comprise additional working memories (additional processors and memories not individually illustrated) including one or more graphics processing units (GPUs) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 520 may be implemented as one or more general purpose processors (preferably having multiple cores) without necessarily departing from the spirit and scope of the present invention.

While the present invention has been particularly described with respect to the illustrated embodiments, it will be appreciated that various alterations, modifications, and adaptations may be made based on the present disclosure and are intended to be within the scope of the present invention. While the invention has been described in connection with what are presently considered to be the most practical and preferred embodiments, it is to be understood that the present invention is not limited to the disclosed embodiments but, on the contrary, is intended to cover various modifications and equivalent arrangements included within the scope of the underlying principles of the invention as described by the various embodiments referenced above and below.

### Selected Embodiments

Embodiment 1. A method of designing a hybrid clinical trial including an external control arm (ECA) study located in at least one site, to support a randomized clinical trial (RCT) study for a treatment of a condition, the method comprising: (1) administering the treatment to at least some of a plurality of RCT participants, wherein each RCT participant is an individual person; (2) detecting at least one outlier ECA candidate in a plurality of candidate records in an ECA candidate database located in the at least one site, wherein each candidate record corresponds to an individual person receiving a standard of care (SOC) treatment for the condition, by calculating a Mahalanobis distance value based on: (i) a point comprising a set of values corresponding to a first plurality of feature variables obtained from the candidate record corresponding to the at least one outlier ECA candidate, wherein each candidate record corresponds to an ECA candidate and comprises information about administering the SOC treatment to the ECA candidate; and (ii) a distribution comprising a plurality of points, wherein each point comprises a set of values corresponding to the first plurality of feature variables obtained from each of a plurality of RCT participant records in a RCT participant database, wherein each participant record corresponds to an RCT participant and comprises information about administering the treatment to the RCT participant; (3) excluding at least one outlier ECA candidate from at least one ECA participant database at the at least one site based on whether the Mahalanobis distance value meets a specified criteria; and (4) dynamically adjusting recruitment into at least one ECA participant database at the at least one site that is recruiting participants into the ECA study by comparing a set of values corresponding to a second set of feature variables obtained from the ECA participant records in at least one ECA participant database to a set of values corresponding to the second set of feature variables obtained from the RCT participant records in the RCT participant database.
Embodiment 2. The method of embodiment 1, further comprising fitting a propensity score model by calculating propensity scores using RCT and ECA participant records to adjust for one or more measured confounders.
Embodiment 3. The method of embodiment 2, wherein the propensity score model is estimated using one or more of a logistic regression model, a machine learning based propensity score model, a probit model, neural networks, support vector machines, decision trees, or meta-classifiers.
Embodiment 4. The method of embodiment 3, wherein propensity scores estimated using the propensity score model are used to match ECA participants to RCT participants based on the one or more measured confounders.
Embodiment 5. The method of any of embodiments 3 and 4, wherein propensity scores estimated using the propensity score model are used to weight ECA and RCT participants based on one or more measured confounders.
Embodiment 6. The method of embodiment 5, wherein the estimated propensity score on at least one ECA participant record is weighted downward if the propensity score model indicates that it is relatively dissimilar to one or more RCT participant records in the RCT participant database, and the estimated propensity score on the at least one RCT participant record is weighted upward if the propensity score model indicates that it is relatively dissimilar to one or more ECA participant records in the ECA participant database.
Embodiment 7. The method of embodiment 6, wherein the propensity scores comprise real numbers greater than or equal to zero and less than or equal to 1.
Embodiment 8. The method of embodiment 7, wherein patient data in the ECA and RCT participant databases is weighted by the propensity score in accordance with an overlap weighting methodology.
Embodiment 9. The method of any of embodiments 7 and 8, wherein patient data in the ECA and RCT participant databases are weighted by the propensity score in accordance with an inverse-probability of treatment weighing (IPTW) methodology.
Embodiment 10. The method of any of embodiments 1-9, wherein the at least one ECA candidate database comprises an electronic health records (EHR) database at a site.
Embodiment 11. The method of any of embodiments 1-10, wherein the at least one ECA candidate database comprises both EHR data and non-EHR data.
Embodiment 12. The method of embodiment 11, wherein the non-EHR data comprises a clinical database at a site.
Embodiment 13. The method of any of embodiments 11 and 12, wherein the non-EHR data comprises a Patient Reported Outcomes (PROs) database.
Embodiment 14. The method of any of embodiments 1-13, wherein dynamically adjusting recruitment from at least one site recruiting participants into the ECA comprises adding one or more ECA candidate records from at least one site-specific ECA candidate database to at least one ECA participant database when an imbalance is identified in the comparison of the set of values corresponding to the second set of feature variables obtained from the ECA participant records in the at least one ECA participant database and the set of values corresponding to the second set of feature variables obtained from the RCT participant records in the RCT participant database, wherein the imbalance is corrected to within a balancing range when the one or more ECA candidate records are added into the at least one ECA participant database.
Embodiment 15. The method of any of embodiments 1-14, wherein the step of dynamically adjusting recruitment from at least one site recruiting participants into the ECA is performed at periodic time intervals for a time duration of the hybrid clinical trial.
Embodiment 16. The method of embodiment 15, wherein the periodic time interval is at least monthly.
Embodiment 17. The method of any of embodiments 14-16, wherein identifying the imbalance in the comparison of the set of values corresponding to the second set of feature variables obtained from the ECA participant records in the at least one ECA participant database and the set of values corresponding to the second set of feature variables obtained from the RCT participant records in the RCT participant database comprises: (1) calculating an absolute standardized mean difference (aSMD) metric between at least one feature variable of the RCT participant records in the RCT participant database and the aSMD metric for at least one feature variable of the ECA participant records across the ECA participant databases of the sites in the ECA study after propensity score adjustments; and (2) identifying an imbalance when the aSMD metric between at least one feature variable of the RCT participant records in the RCT participant database and the at least one feature variable of the ECA participant records across the ECA participant databases of the sites in the ECA study is greater than a threshold value.
Embodiment 18. The method of any of embodiments 14-17, wherein the threshold value is at least 0.10.
Embodiment 19. The method of any of embodiments 14-18, wherein the adjusting the imbalance within the balancing range comprises: (1) contacting the at least one site wherein the aSMD metric between the RCT participant records in the RCT participant database and the ECA participant records at the site indicates an imbalance; and (2) adding one or more ECA candidate records from the ECA candidate database at the at least one site into the at least one ECA participant database at the at least one site, wherein the set of values corresponding to the second set of feature variables obtained from the one or more ECA candidate records in the at least one ECA candidate database at the at least one site, when combined with the ECA participant records for the at least one ECA participant database at the at least one site, are in balance with the set of values corresponding to the second set of feature variables obtained from the RCT participant records in the RCT participant database.
Embodiment 20. A computer program product comprising a non-transitory computer readable medium comprising processor-executable instructions that, when executed by one or more processors, perform the method of any of embodiments 1-19.
Embodiment 21. A computer system comprising one or more processors coupled to the non-transitory computer readable medium of embodiment 20.

1. A method of designing a hybrid clinical trial including an external control arm (ECA) study located in at least one site, to support a randomized clinical trial (RCT) study for a treatment of a condition, the method comprising:
administering the treatment to at least some of a plurality of RCT participants, wherein each RCT participant is an individual person;
detecting at least one outlier ECA candidate in a plurality of candidate records in an ECA candidate database located in the at least one site, wherein each candidate record corresponds to an individual person receiving a standard of care (SOC) treatment for the condition, by calculating a Mahalanobis distance value based on:
a point comprising a set of values corresponding to a first plurality of feature variables obtained from the candidate record corresponding to the at least one outlier ECA candidate, wherein each candidate record corresponds to an ECA candidate and comprises information about administering the SOC treatment to the ECA candidate; and
a distribution comprising a plurality of points, wherein each point comprises a set of values corresponding to the first plurality of feature variables obtained from each of a plurality of RCT participant records in a RCT participant database, wherein each participant record corresponds to an RCT participant and comprises information about administering the treatment to the RCT participant;
excluding at least one outlier ECA candidate from at least one ECA participant database at the at least one site based on whether the Mahalanobis distance value meets a specified criteria; and
dynamically adjusting recruitment into at least one ECA participant database at the at least one site that is recruiting participants into the ECA study by comparing a set of values corresponding to a second set of feature variables obtained from the ECA participant records in at least one ECA participant database to a set of values corresponding to the second set of feature variables obtained from the RCT participant records in the RCT participant database.

2. The method of claim 1, further comprising fitting a propensity score model by calculating propensity scores using RCT and ECA participant records to adjust for one or more measured confounders.

3. The method of claim 2, wherein the propensity score model is estimated using one or more of a logistic regression model, a machine learning based propensity score model, a probit model, neural networks, support vector machines, decision trees, or meta-classifiers.

4. The method of claim 3, wherein propensity scores estimated using the propensity score model are used to match ECA participants to RCT participants based on the one or more measured confounders.

5. The method of claim 3, wherein propensity scores estimated using the propensity score model are used to weight ECA and RCT participants based on one or more measured confounders.

6. The method of claim 5, wherein the estimated propensity score on at least one ECA participant record is weighted downward if the propensity score model indicates that it is relatively dissimilar to one or more RCT participant records in the RCT participant database, and the estimated propensity score on the at least one RCT participant record is weighted upward if the propensity score model indicates that it is relatively dissimilar to one or more ECA participant records in the ECA participant database.

7. The method of claim 6, wherein the propensity scores comprise real numbers greater than or equal to zero and less than or equal to 1.

8. The method of claim 7, wherein patient data in the ECA and RCT participant databases is weighted by the propensity score in accordance with an overlap weighting methodology.

9. The method of claim 7, wherein patient data in the ECA and RCT participant databases are weighted by the propensity score in accordance with an inverse-probability of treatment weighing (IPTW) methodology.

10. The method of claim 1, wherein the at least one ECA candidate database comprises an electronic health records (EHR) database at a site.

11. The method of claim 1, wherein the at least one ECA candidate database comprises both EHR data and non-EHR data.

12. The method of claim 11, wherein the non-EHR data comprises a clinical database at a site.

13. The method of claim 11, wherein the non-EHR data comprises a Patient Reported Outcomes (PROs) database.

14. The method of claim 1, wherein dynamically adjusting recruitment from at least one site recruiting participants into the ECA comprises adding one or more ECA candidate records from at least one site-specific ECA candidate database to at least one ECA participant database when an imbalance is identified in the comparison of the set of values corresponding to the second set of feature variables obtained from the ECA participant records in the at least one ECA participant database and the set of values corresponding to the second set of feature variables obtained from the RCT participant records in the RCT participant database, wherein the imbalance is corrected to within a balancing range when the one or more ECA candidate records are added into the at least one ECA participant database.

15. The method of claim 1, wherein the step of dynamically adjusting recruitment from at least one site recruiting participants into the ECA is performed at periodic time intervals for a time duration of the hybrid clinical trial.

16. The method of claim 15, wherein the periodic time interval is at least monthly.

17. The method of claim 14, wherein identifying the imbalance in the comparison of the set of values corresponding to the second set of feature variables obtained from the ECA participant records in the at least one ECA participant database and the set of values corresponding to the second set of feature variables obtained from the RCT participant records in the RCT participant database comprises:
calculating an absolute standardized mean difference (aSMD) metric between at least one feature variable of the RCT participant records in the RCT participant database and the aSMD metric for at least one feature variable of the ECA participant records across the ECA participant databases of the sites in the ECA study after propensity score adjustments; and
identifying an imbalance when the aSMD metric between at least one feature variable of the RCT participant records in the RCT participant database and the at least one feature variable of the ECA participant records across the ECA participant databases of the sites in the ECA study is greater than a threshold value.

18. The method of claim 17, wherein the threshold value is at least 0.10.

19. The method of claim 14, wherein the adjusting the imbalance within the balancing range comprises:
contacting the at least one site wherein the aSMD metric between the RCT participant records in the RCT participant database and the ECA participant records at the site indicates an imbalance; and
adding one or more ECA candidate records from the ECA candidate database at the at least one site into the at least one ECA participant database at the at least one site,
wherein the set of values corresponding to the second set of feature variables obtained from the one or more ECA candidate records in the at least one ECA candidate database at the at least one site, when combined with the ECA participant records for the at least one ECA participant database at the at least one site, are in balance with the set of values corresponding to the second set of feature variables obtained from the RCT participant records in the RCT participant database.

20. A non-transitory computer readable medium comprising processor-executable instructions that, when executed by one or more processors, perform a method of designing a hybrid clinical trial including an external control arm (ECA) study located in at least one site to support a randomized clinical trial (RCT) study for a treatment of a condition, the method comprising:
administering the treatment to at least some of a plurality of RCT participants, wherein each RCT participant is an individual person;
detecting at least one outlier ECA candidate in a plurality of candidate records in an ECA candidate database located in the at least one site, wherein each candidate record corresponds to an individual person receiving a standard of care (SOC) treatment for the condition, by calculating a Mahalanobis distance value based on:
a point comprising a set of values corresponding to a first plurality of feature variables obtained from the candidate record corresponding to the at least one outlier ECA candidate, wherein each candidate record corresponds to an ECA candidate and comprises information about administering the SOC treatment to the ECA candidate; and
a distribution comprising a plurality of points, wherein each point comprises a set of values corresponding to the first plurality of feature variables obtained from each of a plurality of RCT participant records in a RCT participant database, wherein each participant record corresponds to an RCT participant and comprises information about administering the treatment to the RCT participant;
excluding at least one outlier ECA candidate from at least one ECA participant database at the at least one site based on whether the Mahalanobis distance value meets a specified criteria; and
dynamically adjusting recruitment into at least one ECA participant database at the at least one site that is recruiting participants into the ECA study by comparing a set of values corresponding to a second set of feature variables obtained from the ECA participant records in at least one ECA participant database to a set of values corresponding to the second set of feature variables obtained from the RCT participant records in the RCT participant database.

21. A computer system comprising one or more processors coupled to the non-transitory computer readable medium of claim 20.
